(19) United States
(12) Patent Application Publication
(10) Pub. No.: US 2022/0108780 A1
(43) Pub. Date: Apr. 7, 2022

(54) TRAINING SUPPORT SYSTEM, TRAINING SUPPORT METHOD, AND TRAINING SUPPORT PROGRAM

(71) Applicant: TOYOTA JIDOSHA KABUSHIKI KAISHA, Toyota-shi Aichi-ken (JP)

(72) Inventors: Hisataka Yuasa, Nagoya-shi Aichi-ken (JP); Issei Nakashima, Toyota-shi Aichi-ken (JP); Takeru Fukagawa, Chiryu-shi Aichi-ken (JP); Takuya Iwata, Toyota-shi Aichi-ken (JP); Takashi Shimizu, Nisshin-shi Aichi-ken (JP); Yohei Otaka, Kariya-shi Aichi-ken (JP); Satoshi Hirano, Nagoya-shi Aichi-ken (JP); Masaki Katoh, Nagoya-shi Aichi-ken (JP); Daisuke Imoto, Nagoya-shi Aichi-ken (JP); Shin Kitamura, Nagoya-shi, Aichi-ken (JP); Yoko Inamoto, Nagoya-shi Aichi-ken (JP)

(21) Appl. No.: 17/488,514

(22) Filed: Sep. 29, 2021

(30) Foreign Application Priority Data
Oct. 2, 2020 (JP) 2020-168109

(51) Int. Cl. G16H 20/30; G09B 5/02; G16H 10/60; G16H 15/00; G16H 50/70; G06F 16/22; A63B 24/00

(52) U.S. Cl. CPC G16H 20/30; G09B 5/02; G16H 10/60; A63B 2214/00; G16H 50/70; G06F 16/22; A63B 24/0075; G16H 15/00

(57) ABSTRACT

A training support system including an information acquisition unit configured to acquire trainee information including a symptom of a trainee, a database configured to store a plurality of combinations of training experienced person information including a symptom of a training experienced person, a training performance, and a recovery status, a selection unit configured to select the training experienced person with the symptom similar to the symptom of the trainee acquired by the information acquisition unit from among a plurality of the training experienced persons, and an output unit configured to extract, from the database, at least any of the training performance and the recovery status of the training experienced person selected by the selection unit and then output at least any of the training performance and the recovery status.

### CROSS REFERENCE TO RELATED APPLICATIONS

This application is based upon and claims the benefit of priority from Japanese patent application No. 2020-168109, filed on Oct. 2, 2020, the disclosure of which is incorporated herein in its entirety by reference.

### BACKGROUND

The present disclosure provides a training support system, a training support method, and a training support program.

Japanese Patent No. 6052234 discloses a walking training apparatus for training a user to walk.

### SUMMARY

Recently, in addition to the walking training apparatus disclosed in Japanese Patent No. 6052234, various training systems have been developed for performing training for the purpose of functional recovery of a trainee's physical action. Under such a circumstances, it has become difficult for the trainee to select a training item suitable for him/her from among a large number of training items and then execute it.

The present disclosure has been made in view of the above circumstances, and an object of the present disclosure is to provide a training support system, a training support method, and a training support program which output a training performance of a training experienced person with a symptom similar to that of a trainee and a recovery status of the symptom to thereby enable the trainee to perform effective training.

An example aspect of the present disclosure is a training support system including: an information acquisition unit configured to acquire trainee information including a symptom of a trainee; a database configured to store a plurality of combinations of training experienced person information including a symptom of a training experienced person, a training performance, and a recovery status; a selection unit configured to select the training experienced person with the symptom similar to the symptom of the trainee acquired by the information acquisition unit from among a plurality of the training experienced persons; and an output unit configured to extract, from the database, at least any of the training performance and the recovery status of the training experienced person selected by the selection unit and then output at least any of the training performance and the recovery status. Since this training support system can output the training performance of the training experienced person with the symptom similar to that of the trainee and the recovery status of the symptom, the trainee can perform effective training by referring to the information.

The output unit is a display apparatus configured to display at least any of the training performance and the recovery status of the training experienced person extracted from the database. In this manner, the training item suitable for the recovery of the symptom of the trainee becomes clearer.

The selection unit is configured to select one or more training experienced persons who have the symptoms showing a similarity of a predetermined value or higher to the symptom of the trainee acquired by the information acquisition unit from among the plurality of training experienced persons, and the output unit is configured to extract from the database at least any of the training performance and the recovery status of each of the one or more training experienced persons selected by the selection unit and output at least any of the training performance and the recovery status.

The selection unit is configured to select a predetermined number of the training experience persons from among the plurality of training experienced persons in order, from the highest to the lowest, of the similarity of the symptom to the symptom of the trainee acquired by the information acquisition unit, and the output unit is configured to extract from the database at least any of the training performance and the recovery status of each of the predetermined number of the training experienced persons selected by the selection unit and output at least any of the training performance and the recovery status.

When two or more of the training experienced persons are selected by the selection unit, the output unit is configured to preferentially output at least any of the training performance and the recovery status of each of the two or more of the training experienced persons selected by the selection unit showing a high similarity to the trainee.

The trainee information may further include information about at least any of an age, a gender, a harness used, and an assisting device used of the trainee, in the database, each piece of the training experienced person information may further include information about at least any of an age, a gender, a harness used, and an assisting device used of the training experienced person, and the selection unit may be configured to select, from among the plurality of training experienced persons, the training experienced person with the symptom similar to the symptom of the trainee acquired by the information acquisition unit and also with at least any of the age, the gender, the harness used, and the assisting device used similar to the age, the gender, the harness used, and the assisting device of the trainee, respectively. It is thus possible to improve the accuracy of determining whether or not the trainee is similar to the training experienced person stored in the database.

Another example aspect of the present disclosure is a training support method including: acquiring trainee information including a symptom of a trainee; selecting a training experienced person with the symptom similar to the symptom of the trainee acquired in the acquiring from among a plurality of the training experienced persons in a database configured to store a plurality of combinations of training experienced person information including a symptom of the training experienced person, a training performance, and a recovery status; and extracting, from the database, at least any of the training performance and the recovery status of the training experienced person selected in the selecting and then outputting at least any of the training performance and the recovery status. Since this training support method can output the training performance of the training experienced person with the symptom similar to that of the trainee and the recovery status of the symptom, the trainee can perform effective training by referring to the information.

Another example aspect of the present disclosure is a training support program causing a computer to execute: processing of acquiring trainee information including a symptom of a trainee; processing of selecting a training experienced person with the symptom similar to the symptom of the trainee acquired in the acquiring from among a plurality of the training experienced persons in a database configured to store a plurality of combinations of training experienced person information including a symptom of the training experienced person, a training performance, and a recovery status; and processing of extracting, from the database, at least any of the training performance and the recovery status of the training experienced person selected in the selecting and then outputting at least any of the training performance and the recovery status. Since this training support program can output the training performance of the training experienced person with the symptom similar to that of the trainee and the recovery status of the symptom, the trainee can perform effective training by referring to the information.

According to the present disclosure, it is possible to provide a training support system, a training support method, and a training support program which output a training performance of a training experienced person with a symptom similar to that of a trainee and a recovery status of the symptom to thereby enable the trainee to perform effective training.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration example of a training support system according to a first embodiment;

FIG. 2 is a flowchart showing an operation of the training support system shown in FIG. 1; and

FIG. 3 shows an example of a screen displayed on a display apparatus.

### DESCRIPTION OF EMBODIMENTS

Although the present disclosure is described below through the embodiments, the disclosure according to the claims is not limited to the following embodiments. In addition, not all of the configurations described in the embodiments are indispensable solving the problems. For clarity of description, the following descriptions and drawings are been omitted and simplified as appropriate. In each drawing, the same elements are denoted by the same reference signs and repeated description is omitted as necessary.

### First Embodiment

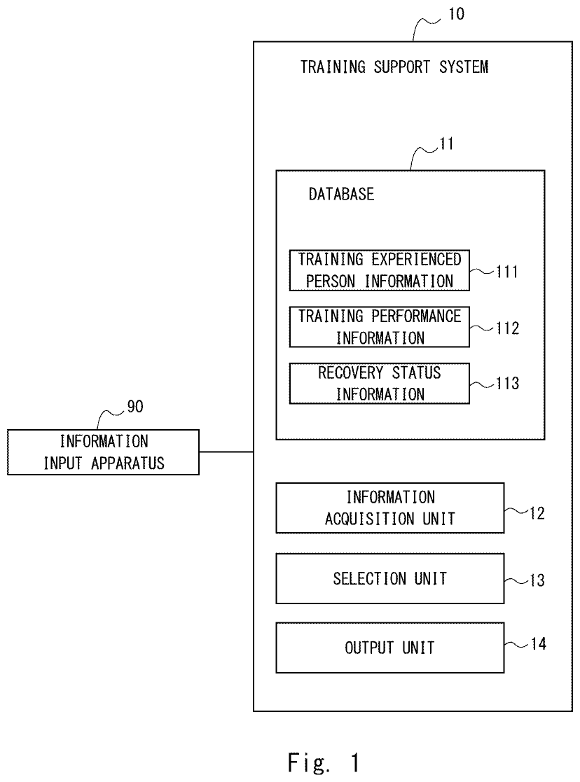

FIG. 1 is a block diagram showing a configuration example of a training support system 10 according to a first embodiment. In FIG. 1, in addition to the training support system 10, an information input apparatus 90 is also shown. The training support system 10 allows a trainee (a subject) to perform effective training by presenting a training performance of a training experienced person with a symptom similar to a symptom of a trainee and a recovery status of the symptom.

Hereinafter, a trainee who intends to perform training using the training support system 10 will be referred to as a “trainee”, and a trainee who has performed training using the training support system 10 or the like will be referred to as a “training experienced person”. The symptom, an age, a gender, a harness used, an assisting device used, a training performance, and a recovery status of the training experienced person are registered in the database.

Here, the trainees are those who suffer paralysis in a part of their bodies due to, for example, a stroke (cerebrovascular disorder) or spinal cord injury. The trainee's physical action functions deteriorates due to the above disease. In this embodiment, the physical action is an action included in, for example, Activities of Daily Living (ADL), and includes actions such as having a meal, going to the toilet, changing clothes, and walking.

The trainees need to perform several types of training to recover an impaired function of a physical action. Examples of the training in order to recover the functions for performing the physical action of having a meal include training of an eating action and a swallowing action. Examples of the training for recovering the function of performing a toilet action include training of a transfer action for transferring to a bed, a wheelchair, and a toilet seat, and training of an excretion activity in the bathroom. Examples of the training for recovering the function of a walking action include training for maintaining a sitting posture, training for maintaining a standing posture, training for walking on a flat ground, and training for walking on a slope. In addition to actively performing the physical actions as described above, the training may also include passive actions such as massaging, which involves receiving joint or muscle stimulation from a caregiver. In other words, there are a large number of types of training to be performed by the trainee. The training described above is also referred to as rehabilitation. For this reason, the training support system 10 can also be referred to as a rehabilitation support system.

The training support system 10 will be described in detail below.

As shown in FIG. 1, the training support system 10 includes a database 11, an information acquisition unit 12, an extraction unit 13, and an output unit 14.

The database 11 stores, for example, training experienced person information 111, training performance information 112, and recovery status information 113 in association with each other.

The training experienced person information 111 includes information about a plurality of training experienced persons who have performed training using the training support system 10 or the like. In some embodiments, the information of the training experienced person includes at least information about a symptom of the training experienced person, and includes information such as the age, the gender, the harness used, and the assisting device used of the training experienced person. In this embodiment, a case in which the information of the training experienced person includes, in addition to the information about the symptom of the training experienced person, information about the age, the gender, the harness used, and the assisting device used of the training experienced person will be described as an example.

The training performance information 112 includes information about a training performance of each training experienced person of the training experienced person information 111. The training performance is, for example, training details and a training time. The recovery status information 113 includes information about the recovery status of the symptom of each training experienced person of the training experienced information 111. The recovery status is, for example, a period from the start of the training to the end of the training and a degree of recovery of the symptom during the period.

The information acquisition unit 12 is an interface for acquiring trainee information input to the information input apparatus 90 provided outside the training support system 10.

In some embodiments, the trainee information acquired by the information acquisition unit 12 includes at least information about the trainee and includes an age, a gender, a harness used, and an assisting device used of the trainee.

The information input apparatus 90 is an apparatus for receiving information input by, for example, a character input operation using a keyboard, a mouse click operation, or a touch operation of a touch panel performed by the user. The information input apparatus 90 receives a predetermined operation performed by the user and outputs the trainee information set according to the received operation to the training support system 10. In the training support system 10, the information acquisition unit 12 acquires the trainee information output from the information input apparatus 90.

The information input apparatus 90 may be integrated with the training support system 10. Further, the information input apparatus 90 may be an apparatus including an information input/output function such as a smartphone or a tablet separate from the training support system 10, and may be connected to the training support system 10 so that communication is possible between them.

The selection unit 13 selects a training experienced person with a symptom similar to that of the trainee acquired by the information acquisition unit 12 among a plurality of training experienced persons included in the training experienced person information 111 of the database 11.

When the trainee information acquired by the information acquisition unit 12 includes, in addition to the symptom of the trainee, information such as the age, the gender, the harness used, and the assisting device used of the trainee, the selection unit 13 selects the training experienced person with the symptom similar to the symptom of the trainee and also the age, the gender, the harness used, and the assisting device used similar to those of the trainee.

Specifically, the similarity of the symptom of each training experienced person to the symptom of the trainee is scored, and the higher the score, the higher the similarity. Likewise, the similarities of the age, the gender, the harness used, and the assisting device used of each training experienced person with respect to the age, the gender, the harness used, and the assisting device used of the trainee, respectively, are scored, and the higher the score, the higher the similarity. However, the weighting of the score of each item differs according to the importance of each item.

Then, the selection unit 13 selects one or more training experienced persons showing the score of a predetermined value or higher (i.e., the similarity is a predetermined value or higher) from among the plurality of training experienced persons included in the training experienced person information 111 of the database 11. Alternatively, the selection unit 13 may select a predetermined number of training experienced persons from among the plurality of training experienced persons included in the training experienced person information 111 of the database 11 in order, from the highest to the lowest, of the scores of the training experienced persons.

The output unit 14 extracts training performances and recovery statuses of one or more training experienced persons selected by the selection unit 13 from the database 11 and outputs the extracted training performances and recovery statuses. In this embodiment, the case in which the output unit 14 is a display apparatus for displaying the training performances and recovery statuses of one or more training experienced persons selected by the selection unit 13 will be described, but the present disclosure is not limited to this. The output unit 14 may be, for example, a speaker for outputting in audio the training performances and recovery statuses of the one or more training experienced persons selected by the selection unit 13, or a transmission apparatus for transmitting the training performances and recovery statuses of the one or more training experienced persons selected by the selection unit 13 to the display apparatus provided outside the training support system 10.

When a plurality of the training experienced persons are selected by the selection unit 13, the output unit 14 preferentially outputs the respective training performances and recovery statuses of the plurality of training experienced persons from the training performances and recovery statuses of the training experienced persons who show a high similarity to the trainees. In this embodiment, since the output unit 14 is the display apparatus, for example, the output unit 14 displays the training performance and the recovery status of each of the training experienced persons selected by the selection unit 13 in order, from the highest to the lowest, of the similarity to the trainee from the upper side to the lower side of the display screen.

### Operation of Training Support System 10

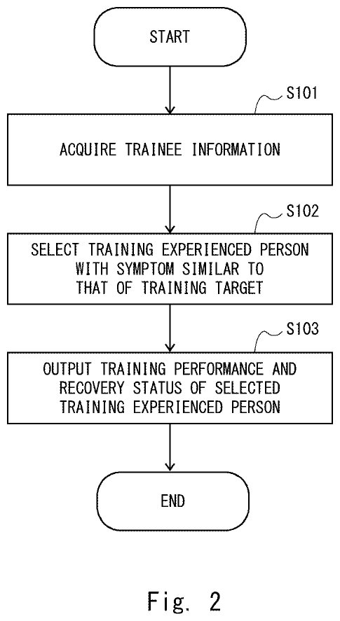

Next, an operation of the training support system 10 will be described with reference to FIG. 2.

FIG. 2 is a flowchart showing the operation of the training support system 10.

Note that it is assumed that training experienced person information 111, training performance information 112, and recovery status information 113 are previously stored in the database 11 in association with each other.

First, the information acquisition unit 12 acquires the trainee information input to the information input apparatus 90 provided outside the training support system 10 (Step S101). In some embodiments, the trainee information includes at least information about the symptom of the trainee and includes the age, the gender, the harness used, and the assisting device used of the trainee.

After that, the selection unit 13 selects the training experienced person with the symptom similar to that of the trainee acquired by the information acquisition unit 12 from among the plurality of training experienced persons included in the training experienced person information 111 of the database 11 (Step S102).

When the trainee information includes, in addition to the symptom of the trainee, information such as the age, the gender, the harness used, and the assisting device used of the trainee, the selection unit 13 selects the training experienced person with the symptom similar to the symptom of the trainee and also the age, the gender, the harness used, and the assisting device used similar to those of the trainee.

After that, the output unit 14 extracts the training performance and the recovery status of the training experienced person selected by the selection unit 13 from the database 11 and outputs them (Step S103). In this embodiment, since the output unit 14 is the display apparatus, the output unit 14 extracts, from the database 11, the training performance and recovery status of the training experienced person selected by the selection unit 13 and displays them.

### Example of the Display Screen

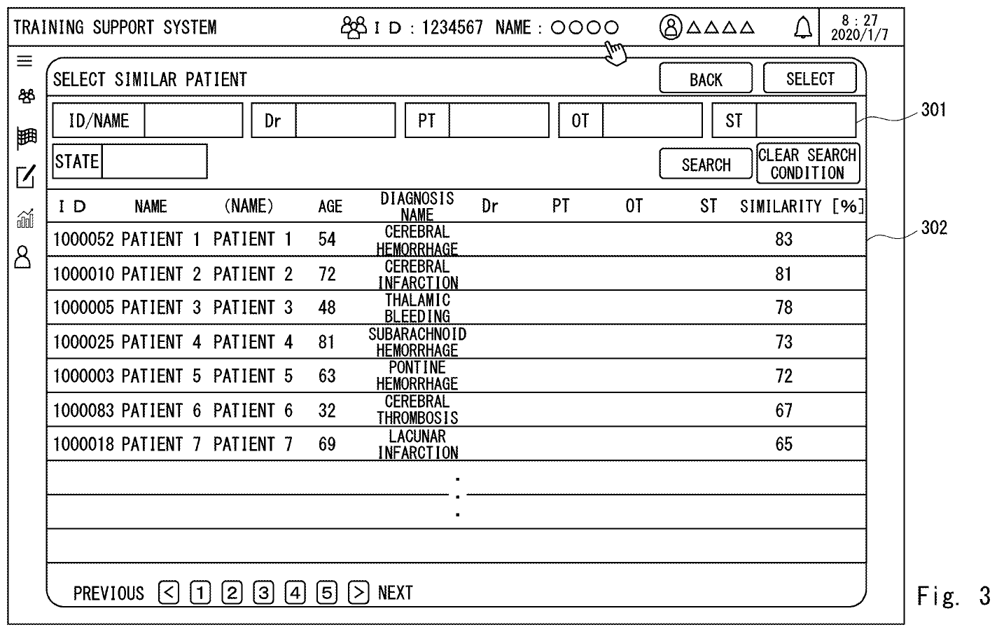

FIG. 3 shows an example of a screen displayed on the display apparatus (the output unit 14).

In the example of FIG. 3, trainee information 301 and information 302 of a plurality of the training experienced persons determined to be similar to the trainee are displayed. Specifically, as the information 302 of the plurality of training experienced persons, the names, ages, diagnosis names (symptoms), etc. of seven training experienced persons are displayed. In addition, pieces of the information of the seven training experienced persons are displayed in order, from the highest to the lowest, of the similarity to the trainee from the upper side to the lower side of the screen. For example, by selecting any one piece of the information of the seven training experienced persons displayed on the screen, the training performance and the recovery status of the selected training experienced person are displayed.

As described above, since the training support system 10 according to this embodiment can output the training performance of the training experienced person with the symptom similar to that of the trainee and the recovery status of the symptom, the trainee can perform effective training by referring to the information.

In the embodiment described above, an example in which the training support system 10 outputs both the training performance of the training experienced person with the symptom similar to that of the trainee and the recovery status of the symptom has been described, but the present disclosure is not limited to this. The training support system 10 may be configured to output at least any of the training performance of the training experienced person with the symptom similar to that of the trainee and the recovery status of the symptom.

Further, although the present disclosure has been described as a hardware configuration in the above embodiments, the present disclosure is not limited to this. The present disclosure can be realized by causing a CPU (Central Processing Unit) to execute a computer program for control processing by the training support system 10.

The above program includes instructions (or software codes) that, when loaded into a computer, cause the computer to perform one or more of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A training support system comprising:
an information acquisition unit configured to acquire trainee information including a symptom of a trainee;
a database configured to store a plurality of combinations of training experienced person information including a symptom of a training experienced person, a training performance, and a recovery status;
a selection unit configured to select the training experienced person with the symptom similar to the symptom of the trainee acquired by the information acquisition unit from among a plurality of the training experienced persons; and
an output unit configured to extract, from the database, at least any of the training performance and the recovery status of the training experienced person selected by the selection unit and then output at least any of the training performance and the recovery status.

2. The training support system according to claim 1, wherein
the output unit is a display apparatus configured to display at least any of the training performance and the recovery status of the training experienced person extracted from the database.

3. The training support system according to claim 1, wherein
the selection unit is configured to select one or more training experienced persons who have the symptoms showing a similarity of a predetermined value or higher to the symptom of the trainee acquired by the information acquisition unit from among the plurality of training experienced persons, and
the output unit is configured to extract, from the database, at least any of the training performance and the recovery status of each of the one or more training experienced persons selected by the selection unit and output at least any of the training performance and the recovery status.

4. The training support system according to claim 1, wherein
the selection unit is configured to select a predetermined number of the training experience persons from among the plurality of training experienced persons in order, from the highest to the lowest, of the similarity of the symptom to the symptom of the trainee acquired by the information acquisition unit, and
the output unit is configured to extract, from the database, at least any of the training performance and the recovery status of each of the predetermined number of the training experienced persons selected by the selection unit and output at least any of the training performance and the recovery status.

5. The training support system according to claim 1, wherein
when two or more of the training experienced persons are selected by the selection unit, the output unit is configured to preferentially output at least any of the training performance and the recovery status of each of the two or more of the training experienced persons selected by the selection unit showing a high similarity to the trainee.

6. The training support system according to claim 1, wherein
the trainee information further includes information about at least any of an age, a gender, a harness used, and an assisting device used of the trainee,
in the database, each piece of the training experienced person information further includes information about at least any of an age, a gender, a harness used, and an assisting device used of the training experienced person, and
the selection unit is configured to select, from among the plurality of training experienced persons, the training experienced person with the symptom similar to the symptom of the trainee acquired by the information acquisition unit and also with at least any of the age, the gender, the harness used, and the assisting device used similar to the age, the gender, the harness used, and the assisting device of the trainee, respectively.

7. A training support method comprising:
acquiring trainee information including a symptom of a trainee;
selecting a training experienced person with the symptom similar to the symptom of the trainee acquired in the acquiring from among a plurality of the training experienced persons in a database configured to store a plurality of combinations of training experienced person information including a symptom of the training experienced person, a training performance, and a recovery status; and
extracting, from the database, at least any of the training performance and the recovery status of the training experienced person selected in the selecting and then outputting at least any of the training performance and the recovery status.

8. A non-transitory computer readable medium storing a training support program causing a computer to execute:
processing of acquiring trainee information including a symptom of a trainee;
processing of selecting a training experienced person with the symptom similar to the symptom of the trainee acquired in the acquiring from among a plurality of the training experienced persons in a database configured to store a plurality of combinations of training experienced person information including a symptom of the training experienced person, a training performance, and a recovery status; and
processing of extracting, from the database, at least any of the training performance and the recovery status of the training experienced person selected in the selecting and then outputting at least any of the training performance and the recovery status.
